(19) United States
(12) Patent Application Publication
(10) Pub. No.: US 2025/0177288 A1
(43) Pub. Date: Jun. 5, 2025

(54) SOLUBLE MICRONEEDLE FOR LOCAL ANESTHESIA AND LOCAL ANESTHESIA PATCH COMPRISING SAME

(71) Applicants: UIF UNIVERSITY INDUSTRY FOUNDATION YONESI UNIVERSITY, Seoul (KR); JUVIC INC., Seoul (KR)

(72) Inventors: Hyungil JUNG, Seoul (KR); Huisuk YANG, Seoul (KR); Geonwoo KANG, Seoul (KR)

(73) Assignees: UIF UNIVERSITY INDUSTRY FOUNDATION YONESI UNIVERSITY, Seoul (KR); JUVIC INC., Seoul (KR)

(21) Appl. No.: 18/862,017

(22) PCT Filed: Apr. 28, 2023
(86) PCT No.: PCT/KR2023/005860

§ 371 Date: Oct. 31, 2024

(30) Foreign Application Priority Data
May 2, 2022 (KR) 10-2022-0054116

(51) Int. Cl. A61K 9/00; A61K 31/167; A61K 47/32; A61K 47/36; A61P 23/02

(52) U.S. Cl. CPC A61K 9/0021; A61K 31/167; A61K 47/32; A61K 47/36; A61P 23/02

(57) ABSTRACT

The present invention relates to a soluble microneedle for local anesthesia and a local anesthesia patch comprising same, the soluble microneedle comprising an amide-based local anesthetic, hyaluronic acid, and polyvinylpyrrolidone in a specific content ratio. The present invention uses the soluble microneedle to implement direct drug delivery by physically permeating through the stratum corneum and mucosa, and thus a precise and rapid anesthetic effect may be exhibited. Especially, the microneedle comprising a specific amount of polyvinylpyrrolidone is dissolved, and the anesthetic contained in the microneedle is rapidly delivered to nerve cells, and thus the disadvantage of a long waiting time of conventional anesthetic creams may be overcome, and thus the present invention has the advantage of being usable in various ways in dental, dermatology, and other surgical procedures requiring local anesthesia.

### TECHNICAL FIELD

The present invention relates to a dissolving microneedle for local anesthesia and a local anesthesia patch including the same, and more particularly, to a dissolving microneedle for local anesthesia and a local anesthesia patch including the same, the dissolving microneedle including an amide-based local anesthetic, hyaluronic acid, and polyvinylpyrrolidone at a specific content ratio, thereby implementing an anesthetic effect precisely and rapidly even with a low dose.

### BACKGROUND ART

Local anesthetics are drugs that act on peripheral sensory nerves to block neural transmission, thereby dulling or eliminating pain sensations in a local area. Clinically, local anesthetics are used for local anesthesia to relieve toothaches or prevent pain during extraction in the dental field and are used for the purpose of local anesthesia during simple surgical operations in the surgical field (Becker D. E. et al., 2012). Representative examples of local anesthetic drugs include cocaine, benzocaine, lidocaine, bupivacaine, tetracaine, procaine, etidocaine, and the like (McLure H. A. et al., 71, 59-74, 2005).

Most local anesthetics have been formulated and developed as injectables (McLure H. A. et al., 71, 59-74, 2005), and while injectables have the advantage of being effective quickly, they have a risk of systemic side effects due to a rapid increase in their concentrations in blood. In addition, since children and some adults avoid getting injections due to fear of injections in some cases, in order to address such a problem, topical pharmaceutical preparations formulated as ointments or gels have been developed and are commercially available.

Recently, patches containing lidocaine, one type of local anesthetic, have been developed, and these patches are intended for attachment to the skin. However, the patches have limitations that systemic side effects may be caused due to difficulty of controlling the dose during the time it takes for the anesthetic effect to occur.

Accordingly, as a result of trying to develop a local anesthesia method that allows an anesthetic effect to be achieved promptly and precisely, the present inventors have completed the present invention by confirming that, when a microneedle including lidocaine, hyaluronic acid, and polyvinylpyrrolidone at a certain content ratio is attached to an area targeted for local anesthesia, as the microneedle is dissolved, the anesthetic contained therein can be promptly delivered to neurons.

### DISCLOSURE

### Technical Problem

The present invention is directed to providing a dissolving microneedle for local anesthesia and a local anesthesia patch including the same.

### Technical Solution

To achieve the above objective, the present invention provides a dissolving microneedle for local anesthesia that includes an amide-based local anesthetic, hyaluronic acid, and polyvinylpyrrolidone.

In the present invention, the amide-based local anesthetic may be one or more selected from the group consisting of lidocaine, mepivacaine, prilocaine, articaine, bupivacaine, etidocaine, and salts thereof.

In the present invention, the amide-based local anesthetic may be lidocaine.

In the present invention, in the dissolving microneedle for local anesthesia, the amide-based local anesthetic, the hyaluronic acid, and the polyvinylpyrrolidone may be included at a weight ratio ranging from 1:1.5:0.05 to 1:2.5:0.3.

In the present invention, in the dissolving microneedle for local anesthesia, the amide-based local anesthetic, the hyaluronic acid, and the polyvinylpyrrolidone may be included at 32 wt %, 63 wt %, and 5 wt %, respectively.

In the present invention, the dissolving microneedle for local anesthesia may be for local anesthesia for treating dental or dermatological diseases.

In the present invention, the dissolving microneedle for local anesthesia may be conical, candle-shaped, or egg-shaped.

In the present invention, the dissolving microneedle for local anesthesia may be made of three layers consisting of a support portion, a middle end portion, and a sharp end portion, and the amide-based local anesthetic, the hyaluronic acid, and the polyvinylpyrrolidone may be included in the middle end portion.

In the present invention, the amide-based local anesthetic, the hyaluronic acid, and the polyvinylpyrrolidone may constitute an entire layer of the middle end portion.

In the present invention, the amide-based local anesthetic, the hyaluronic acid, and the polyvinylpyrrolidone may be located in a core layer of the middle end portion, and the core layer may be formed to be covered by a shell layer.

The present invention also provides a local anesthesia patch including the dissolving microneedle and a support layer configured to support the microneedle.

In the present invention, the support layer may have a thickness ranging from 150 to 500 μm.

In the present invention, the microneedle may have a height ranging from 500 to 1,000 μm.

In the present invention, the local anesthesia patch may contain lidocaine at an administration dose of 1 mg to 5 mg.

In the present invention, the support layer may be a polymer film and may be: (i) a plastic sheet or film selected from the group consisting of polyethylene, polyurethane, polyvinyl alcohol, polypropylene, polyethylene terephthalate, polystyrene (GPPS), polylactide, polyglycolide, polycaprolactone, polyethylene glycol, polyanhydride, polyamide, polyesteramide, polyorthoester, polydioxanone, polyacetal, polyketal, polycarbonate, polyorthocarbonate, polyphosphazene, polyhydroxybutyrate, polyhydroxyvalerate, polyalkylene oxalates, polyalkylene succinates, poly(malic acid), poly(amino acid), poly(methylvinyl ether), chitin, chitosan and its copolymer, terpolymer, carboxymethylcellulose, hyaluronic acid, polyvinylpyrrolidone, and an ethylene vinyl acetate (EVA) copolymer; (ii) a paper sheet selected from the group consisting of sterilization paper, cellophane, non-woven fabric, and woven fabric; (iii) a silicone resin thin film formed by spraying or spreading; or (iv) a fluorine oil thin film formed by spraying or spreading.

### Advantageous Effects

According to the present invention, since direct drug delivery is implemented by a dissolving microneedle physically penetrating the stratum corneum and mucosa, an anesthetic effect can be achieved precisely and rapidly even with a low dose. In particular, since, as the microneedle including a specific amount of polyvinylpyrrolidone is dissolved, the anesthetic contained therein is promptly delivered to neurons, long waiting time, which is a disadvantage of conventional anesthetic creams, can be overcome, and thus there is an advantage that the present invention can be utilized in various ways in dental and dermatological treatments and other surgical procedures that require local anesthesia.

### DESCRIPTION OF DRAWINGS

FIG. 1 shows results of a microneedle shape test of a lidocaine-containing dissolving microneedle (Li-DMN) patch according to one embodiment of the present invention.

FIG. 2 shows results of a microbial limit test of the Li-DMN patch according to one embodiment of the present invention.

FIG. 3 shows results of a skin irritation test of the Li-DMN patch according to one embodiment of the present invention.

FIG. 4 shows a test method using the von Frey test (VFT) for testing anesthetic efficacy in the present invention. (See Quantitative assessment of tactile allodynia in the rat paw, S R Chaplan et al., J Neurosci Methods. 1994 July; 53 (1): 55-63.)

FIG. 5 shows results of the VFT of the Li-DMN patch according to one embodiment of the present invention. (Mean±Std; †p<0.05, comparing 0, 10, and 20 minutes after application of the lidocaine patch; **p<0.01, ***p<0.001, comparing the untreated group and the group treated with the lidocaine patch for the same times.)

FIG. 6 shows results of applying Li-DMN patches containing lidocaine at various doses to gums and confirming the concentration of lidocaine delivered to the gums in order to determine the dose of lidocaine to be administered using the Li-DMN patch according to one embodiment of the present invention (n=5, Mean±Std Err).

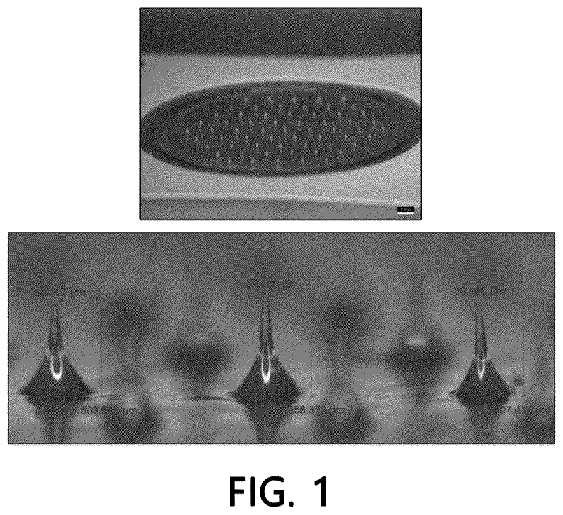

### MODES OF THE INVENTION

Unless otherwise defined, all technical and scientific terms used herein have the same meanings as those commonly understood by those skilled in the art to which the present invention pertains. Generally, the nomenclature used herein is well known and commonly employed in the art.

Unless otherwise indicated, all numbers expressing sizes, amounts, and physical properties used in the present specification and the claims should be understood as being preceded in all instances by the term “about.” Accordingly, unless indicated to the contrary, the numerical parameters set forth in the present specification and the appended claims are approximations that can vary depending on the desired properties sought to be obtained by a person of ordinary skill in the art using the teachings disclosed in the present specification. Preferably, “about” may mean±10% of the stated value.

In the present invention, it has been confirmed that a local anesthetic controlled to be a precise dose can be immediately delivered when a dissolving microneedle including an amide-based local anesthetic (e.g., lidocaine), hyaluronic acid, and polyvinylpyrrolidone is fabricated and the dissolving microneedle is used as a method of delivering a local anesthetic to tissue under the stratum corneum through the epidermis.

Accordingly, one aspect of the present invention relates to a dissolving microneedle for local anesthesia that includes an amide-based local anesthetic, hyaluronic acid, and polyvinylpyrrolidone.

In the present invention, the amide-based local anesthetic may be one or more selected from the group consisting of lidocaine, mepivacaine, prilocaine, articaine, bupivacaine, etidocaine, and salts thereof, but the present invention is not limited thereto.

In the present invention, the dissolving microneedle for local anesthesia may include the amide-based local anesthetic, the hyaluronic acid, and the polyvinylpyrrolidone at a content ratio ranging from 1:1.5:0.05 to 1:2.5:0.3, preferably at a content ratio from 1:1.7:0.1 to 1:2.3:0.25, and most preferably at a content ratio of about 1:2:0.15, but the present invention is not limited thereto.

In the present invention, when the dissolving microneedle for local anesthesia is made of an amide-based local anesthetic, hyaluronic acid, and polyvinylpyrrolidone, the amide-based local anesthetic may be included at 30 to 34 wt %, the hyaluronic acid may be included at 61 to 65 wt %, and the polyvinylpyrrolidone may be included at 1 to 9 wt %. Preferably, the amide-based local anesthetic may be included at 31 to 33 wt %, the hyaluronic acid may be included at 62 to 64 wt %, and the polyvinylpyrrolidone may be included at 3 to 7 wt %, and most preferably, the amide-based local anesthetic may be included at 32 wt %, the hyaluronic acid may be included at 63 wt %, and the polyvinylpyrrolidone may be included at 5 wt %, but the present invention is not limited thereto.

In the present invention, the amide-based local anesthetic may be lidocaine, but the present invention is not limited thereto.

In the present invention, preferably, the lidocaine may be lidocaine hydrochloride. In the present invention, the hyaluronic acid is an excipient that maintains the form of the microneedle, and preferably, the hyaluronic acid may be included at 62 to 64 wt % relative to the total weight of the microneedle in order to allow the dissolving microneedle to have sufficient strength to physically penetrate the skin and mucosa in addition to having the properties of being biocompatible and biodegradable.

In the present invention, preferably, the polyvinylpyrrolidone may be included at 3 to 7 wt % relative to the total weight of the microneedle in order to enhance a degradation speed to be suitable especially for the microneedle according to the present invention.

In the present invention, the dissolving microneedle for local anesthesia may be for local anesthesia for treating dental or dermatological diseases, but the present invention is not limited thereto.

In the present invention, the dissolving microneedle for local anesthesia may be conical, candle-shaped, or egg-shaped, but the present invention is not limited thereto.

In the present invention, the microneedle may be fabricated according to a method described in Korean Patent Registration No. 10-1590172 (Method of preparing microstructure using centrifugal force and microstructure prepared by the same), but the present invention is not limited thereto. For example, the microneedle may be fabricated according to a method described in Korean Patent Registration No. 10-1853308, Korean Patent Registration No. 10-1808066, Korean Patent Registration No. 10-2198478, Korean Patent Registration No. 10-1827739, Korean Patent Registration No. 10-1754309, Korean Patent Registration No. 10-1488397, or Korean Patent Registration No. 10-1527469.

In the present invention, the microneedle may have a height ranging from 500 to 1,000 μm, but the present invention is not limited thereto. When the present invention is applied with the height of the microneedle set to range from 500 to 1,000 μm, there is an advantage that the microneedle can be inserted at a sufficient depth into the skin or mucosa, and an anesthetic drug contained in the microneedle can be effectively delivered.

In the present invention, a lower end portion of the microneedle may have a diameter ranging from 200 to 800 μm, but the present invention is not limited thereto.

In the present invention, a sharp end portion of the microneedle may have a diameter ranging from 1 to 60 μm, but the present invention is not limited thereto.

Hyaluronic acid is one kind of glycosaminoglycan (mucopolysaccharide) and has a structure in which disaccharide units of N-acetyl glucosamine and glucuronic acid are connected. Examples of hyaluronic acid may include bio-based hyaluronic acid isolated from a cockscomb, an umbilical cord, or the like and culture-based hyaluronic acid mass-produced using Lactobacillus, Streptococcus, or the like. Regarding bio-based hyaluronic acid, collagen contained in the living organism from which the bio-based hyaluronic acid is derived cannot be completely removed, and the remaining collagen may have a negative effect. Thus, culture-based hyaluronic acid that does not contain collagen is preferable. Therefore, preferably, hyaluronic acid may include culture-based hyaluronic acid at 50 mass % or more.

In fabricating a microneedle using hyaluronic acid or a derivative thereof, in a microneedle array formed using such a high molecular material, when the weight average molecular weight decreases, the microneedle array becomes hard and it becomes easy to insert the microneedle array into an application site, and conversely, when the weight average molecular weight increases, since the mechanical strength of the microneedle array is improved and stickiness of the microneedle array increases, the microneedle array tends to become flexible and easier to apply to curved areas such as the gums. For the objective of the present invention, preferably, the weight average molecular weight may range from 5,000 to 2,000,000.

In the present invention, the dissolving microneedle for local anesthesia may include an additional excipient in addition to the amide-based local anesthetic, the hyaluronic acid, and the polyvinylpyrrolidone.

In the present invention, in a case in which the dissolving microneedle for local anesthesia includes the additional excipient, when based on the total weight of the amide-based local anesthetic, the hyaluronic acid, and the polyvinylpyrrolidone excluding the additional excipient, the amide-based local anesthetic may be included at 30 to 34 wt %, the hyaluronic acid may be included at 61 to 65 wt %, and the polyvinylpyrrolidone may be included at 1 to 9 wt %. Preferably, the amide-based local anesthetic may be included at 31 to 33 wt %, the hyaluronic acid may be included at 62 to 64 wt %, and the polyvinylpyrrolidone may be included at 3 to 7 wt %, and most preferably, the amide-based local anesthetic may be included at 32 wt %, the hyaluronic acid may be included at 63 wt %, and the polyvinylpyrrolidone may be included at 5 wt %.

The additional excipient may be present preferably in an amount of 2 wt % or more, more preferably in an amount of 5 wt % or more, and most preferably in an amount of 10 wt % or more based on the total weight of the microneedle. In addition, the additional excipient may be present preferably in an amount of 98 wt % or less, more preferably in an amount of 90 wt % or less, and most preferably in an amount of 75 wt % or less or 50 wt % or less based on the total weight of the microneedle. In some embodiments, the microneedle may include one or more additional excipients preferably at 10 to 75 wt % or 10 to 50 wt %, and wt % is based on the total content of the microneedle.

Examples of the additional excipient may include a buffering agent, a carbohydrate, a polymer, an amino acid, a peptide, a surfactant, a protein, a nonvolatile nonaqueous solvent, an acid, a base, an antioxidant, and saccharin.

One or more buffering agents may be used as some of the one or more additional excipients. A buffering agent may generally serve to stabilize pH in a step of fabricating the dissolving microneedle. A specific buffering agent that is used may be appropriately selected by a person skilled in the art depending on the contents of lidocaine, hyaluronic acid, and polyvinylpyrrolidone of the present invention.

Examples of the buffering agent may include histidine, phosphate buffer, acetate buffer, citrate buffer, glycine buffer, ammonium acetate buffer, succinate buffer, pyrophosphate buffer, Tris acetate (TA) buffer, and Tris buffer. A buffered saline solution may also be used as the buffering agent. Examples of the buffered saline solution include phosphate buffered saline (PBS), Tris buffered saline (TBS), saline-sodium acetate buffer (SSA), and saline-sodium citrate buffer (SSC).

One or more carbohydrates, including mixtures of carbohydrates, may be used as some of the one or more additional excipients. Carbohydrates may be sugars, including monosaccharides, disaccharides, and polysaccharides, and examples of the carbohydrates may include non-reducing sugars such as raffinose, stachyose, sucrose, and trehalose; and reducing sugars such as monosaccharides and disaccharides. Examples of the monosaccharides may include apiose, arabinose, digitoxose, fucose, fructose, galactose, glucose, gulose, hamamellose, idose, lyxose, mannose, ribose, tagatose, sorbitol, xylitol, and xylose. Examples of the disaccharides may include sucrose, trehalose, cellobiose, gentiobiose, lactose, lactulose, maltose, melibiose, primeverose, rutinose, scillabiose, sophorose, turanose, and vicianose. In another embodiment, sucrose, trehalose, fructose, maltose, or a combination thereof may be used. Also encompassed by the present invention are all optical isomers (D-isomers, L-isomers, and racemic isomers) of the sugars listed above.

Examples of the polysaccharides may include starches such as hydroxyethyl starch, pregelatinized corn starch, pentastarch, dextrin, dextran or dextran sulfate, gamma-cyclodextrin, alpha-cyclodextrin, beta-cyclodextrin, glucosyl-alpha-cyclodextrin, maltosyl-alpha-cyclodextrin, glucosyl-beta-cyclodextrin, maltosyl-beta-cyclodextrin, 2-hydroxy-beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. According to embodiments, hydroxyethyl starch, dextrin, dextran, gamma-cyclodextrin, beta-cyclodextrin, or combinations thereof may be used. According to embodiments, dextran having a mean molecular mass ranging from 35,000 to 76,000 may be used.

The one or more carbohydrates may be cellulose. Examples of suitable cellulose may include hydroxyethyl cellulose (HEC), methyl cellulose (MC), microcrystalline cellulose, hydroxypropyl methyl cellulose (HPMC), hydroxyethyl methyl cellulose (HEMC), hydroxypropyl cellulose (HPC), and mixtures thereof.

One or more amino acids may be used for at least some of the one or more additional excipients. Examples of suitable amino acids may include lysine, histidine, cysteine, glutamate, lysine acetate, sarcosine, proline, threonine, asparagine, aspartic acid, glutamic acid, glutamine, isoleucine, leucine, methionine, phenylalanine, serum tryptophan, tyrosine, valine, alanine, arginine, and glycine. In many cases, salt forms of amino acids may be used to increase the aqueous solubility of amino acids in aqueous media or formulations.

One or more peptides may be used for at least some of the one or more additional excipients. The amino acids that make up the peptides may be the same, or at least some of them may be different from each other. Examples of suitable polyamino acids (same amino acids) may include polyhistidine, polyaspartic acid, and polylysine.

One or more proteins may be used for at least some of the one or more additional excipients. Examples of suitable proteins may include human serum albumin and bioengineered human albumin.

One or more saccharins may be used for at least some of the one or more additional excipients. For example, saccharin is saccharin sodium dihydrate.

One or more lipids may be used for at least some of the one or more additional excipients. For may be example, the lipid dipalmitoylphosphatidylcholine (DPPC).

One or more acids and/or bases may be used for at least some of the one or more additional excipients. For example, one or more weak acids, weak bases, strong acids, strong bases, or some combination thereof may be used. Acids and bases may serve to solubilize or stabilize the local anesthetic and/or dose-extending component. These acids and bases may be referred to as counterions. These acids and bases may be organic or inorganic. Examples of weak acids include acetic acid, propionic acid, pentanoic acid, citric acid, succinic acid, glycolic acid, gluconic acid, glucuronic acid, lactic acid, malic acid, pyruvic acid, tartaric acid, tartronic acid, fumaric acid, glutamic acid, aspartic acid, malonic acid, butyric acid, crotonic acid, diglycolic acid, and glutaric acid. Examples of strong acids include hydrochloric acid, hydrobromic acid, nitric acid, sulfonic acid, sulfuric acid, maleic acid, phosphoric acid, benzene sulfonic acid, and methane sulfonic acid. Examples of weak bases include ammonia, histidine, lysine, arginine, monoethanolamine, diethanolamine, triethanolamine, tromethamine, methylglucamine, and glucosamine. Examples of strong bases include sodium hydroxide, potassium hydroxide, calcium hydroxide, and magnesium hydroxide.

One or more surfactants may be used for at least some of the one or more additional excipients. The one or more surfactants may be amphoteric, cationic, anionic, or nonionic. Examples of suitable surfactants may include lecithin, polysorbate (such as polysorbate 20, polysorbate 40, and polysorbate 80), glycerol, sodium lauroamphoacetate, sodium dodecyl sulfate, cetylpyridinium chloride (CPC), dodecyltrimethyl ammonium chloride (DoTAC), sodium desoxycholate, benzalkonium chlorides, sorbitan laurate, and alkoxylated alcohols (such as laureth-4).

One or more inorganic salts may be used for at least some of the one or more additional excipients. Examples of suitable inorganic salts may include sodium chloride and potassium chloride.

Nonvolatile nonaqueous solvents may also be used for at least some of the one or more additional excipients. Examples thereof may include propylene glycol, dimethyl sulfoxide, glycerin, 1-methyl-2-pyrrolidinone, N,N-dimethylformamide, and the like.

One or more antioxidants may be used for at least some of the one or more additional excipients. Examples of suitable antioxidants may include sodium citrate, citric acid, ascorbic acid, methionine, sodium ascorbate, and combinations thereof.

In the present invention, the dissolving microneedle for local anesthesia may be made of three layers consisting of a support portion, a middle end portion, and a sharp end portion, and the amide-based local anesthetic, the hyaluronic acid, and the polyvinylpyrrolidone may be included in the middle end portion, but the present invention is not limited thereto.

Preferably, in the present invention, a local anesthetic component may not be contained in the support portion and the sharp end portion.

In the present invention, the support portion may be made of a polymer (e.g., hyaluronic acid) for the microneedle to have sufficient strength to penetrate the skin and deliver the entire amide-based local anesthetic contained therein to the skin, an additive (e.g., polyvinylpyrrolidone) for the support portion to be rapidly dissolved in the skin, and an additive (e.g., triamcinolone) for repairing micro-wounds formed due to the microneedle penetrating the skin.

In the present invention, the sharp end portion may be made of a polymer (e.g., hyaluronic acid) for the sharp end portion to have sufficient strength to physically penetrate the skin layer and an additive (e.g., polyvinylpyrrolidone) for the sharp end portion to be rapidly dissolved in the skin.

In the present invention, the amide-based local anesthetic, the hyaluronic acid, and the polyvinylpyrrolidone may constitute an entire layer of the middle end portion, but the present invention is not limited thereto.

When the amide-based local anesthetic, the hyaluronic acid, and the polyvinylpyrrolidone according to the present invention constitute the entire layer of the middle end portion, an anesthetic component including the amide-based local anesthetic, the hyaluronic acid, and the polyvinylpyrrolidone may be exposed to the outside through a central portion of a side surface of the microneedle.

In the present invention, the amide-based local anesthetic, the hyaluronic acid, and the polyvinylpyrrolidone may be located in a core layer of the middle end portion, and the core layer may be formed to be covered by a shell layer, but the present invention is not limited thereto.

When the amide-based local anesthetic, the hyaluronic acid, and the polyvinylpyrrolidone according to the present invention are located in the core layer, the core layer may physically penetrate the skin layer, may be surrounded by a biocompatible aqueous polymer constituting the sharp end portion and the support portion to allow them to be rapidly dissolved in the skin, and may be embedded in the microneedle without any exposed portion.

In one embodiment, the microneedle according to the present invention may be formed in the shape of a structure described in Korean Patent Publication No. 10-2022-0003800. Accordingly, the content described in the above-mentioned Korean publicized patent is incorporated by reference into the present specification as one embodiment for implementing the present invention. In the above publicized patent, the term “base layer” may be used interchangeably with the support portion of the present invention, and the term “shell layer” may be used interchangeably with the sharp end portion of the present invention.

In one embodiment, the shell layer of the middle end portion of the microneedle of the present invention may be made of the same components as the sharp end portion, but the present invention is not limited thereto.

In addition, the dissolving microneedle of the present invention may be provided in the form of a patch so that the local anesthetic can be easily attached and delivered to the mucosa, gums, or skin.

Accordingly, another aspect of the present invention relates to a local anesthesia patch including the dissolving microneedle and a support layer configured to support the microneedle.

In the present invention, the support layer may have a thickness ranging from 100 μm to 1,000 μm, for example, from 150 μm to 500 μm, but the present invention is not limited thereto.

In the present invention, the microneedle may have a height ranging from 300 μm to 2,000 μm, for example, from 500 μm to 1,000 μm, but the present invention is not limited thereto.

In the present invention, the patch may contain lidocaine at any administration dose of 1 mg to 5 mg. Preferably, the patch may contain lidocaine at an administration dose of 2 mg to 5 mg, in another embodiment, of 1 mg to 4 mg, e.g., of 1.5 mg to 3 mg, more preferably 1.8 mg to 2.5 mg.

In the present invention, the patch may be attached for 1 minute to 20 minutes, e.g., for 1 minute to 10 minutes, preferably for 2 minutes to 10 minutes, more preferably for 2 minutes to 8 minutes, even more preferably for 2 minutes to 6 minutes, and most preferably for 3 minutes to 5 minutes, to an area requiring local anesthesia.

In one embodiment when the patch contains lidocaine at an administration dose of 1 mg to 1.5 mg, the patch may be attached for 3 minutes to 20 minutes to the area requiring local anesthesia.

In another embodiment, when the patch contains lidocaine at an administration dose of 1.5 mg to 3 mg, the patch may be attached for 1 minute to 10 minutes to the area requiring local anesthesia.

In still another embodiment, when the patch contains lidocaine at an administration dose of about 2 mg, the patch may be attached for about 3 minutes to the area requiring local anesthesia.

The application time of the patch according to the administration dose may be appropriately controlled by an expert depending on the sensitivity to lidocaine of an individual requiring local anesthesia, but when the patch according to the present invention is used, a lower dose and/or a shorter application time may produce an equal or greater effect compared to when the currently commercialized lidocaine gel formulation is used.

In the present invention, the support layer may be a polymer film and may be: (i) a plastic sheet or film selected from the group consisting of polyethylene, polyurethane, polyvinyl alcohol, polypropylene, polyethylene terephthalate, polystyrene (GPPS), polylactide, polyglycolide, polycaprolactone, polyethylene glycol, polyanhydride, polyamide, polyesteramide, polyorthoester, polydioxanone, polyacetal, polyketal, polycarbonate, polyorthocarbonate, polyphosphazene, polyhydroxybutyrate, polyhydroxyvalerate, polyalkylene oxalates, polyalkylene succinates, poly(malic acid), poly(amino acid), poly(methylvinyl ether), chitin, chitosan and its copolymer, terpolymer, carboxymethylcellulose, hyaluronic acid, polyvinylpyrrolidone, and an ethylene vinyl acetate (EVA) copolymer; (ii) a paper sheet selected from the group consisting of sterilization paper, cellophane, non-woven fabric, and woven fabric; (iii) a silicone resin thin film formed by spraying or spreading; or (iv) a fluorine oil thin film formed by spraying or spreading, but the present invention is not limited thereto.

In the present invention, the microneedle may be formed in a specific shape (e.g., a conical shape, a candle shape, or an egg shape) after being applied at predetermined intervals on the support layer.

In order to reinforce adhesion of the microneedle to the mucosa, gums, or skin, preferably, the support layer may have stickiness to the mucosa, gums, or skin.

As one form of the support layer having stickiness, a support layer coated with a sticky material, that is, a support layer on which a sticking agent is applied, may be used.

As the sticky material, sticking agents commonly used in patch preparations may be used, and for example, it may be preferable to use an acrylic, silicone, or rubber sticking agent of a grade that has adhesiveness to wet sides.

As another form of the support layer having stickiness, a support body may be aqueous. A low-molecular-weight aqueous film made of polyvinylpyrrolidone (PVP), carboxymethylcellulose (CMC), polyvinyl alcohol (PVA), and the like may be used to exhibit stickiness due to moisture in the mucosa, gums, or skin. In this case, in order to prevent the support layer from being adhered to the side opposite to the mucosa, gums, or skin to which the patch is desired to be applied, a surface facing the opposite side of the aqueous support body may be further laminated with a nonaqueous polymer film.

The microneedle array and the microneedle patch of the present invention may be attached to an area requiring local anesthesia such as the mucosa, gums, or skin, and then a back surface of the microneedle may be pressed so that a local anesthetic is administered to the mucosa, gums, or skin.

In another embodiment, the microneedle may be fabricated in the form of a microneedle array including a plurality of microneedles on a substrate layer and may be attached to the support layer.

That is, the support layer may be integrated with a back surface of the microneedle array using an adhesive or a sticking agent. The sizes of the microneedle array and the support layer may be the same, but in order to reinforce adhesion of the microneedle array to the mucosa, oral cavity, or skin, it may be more preferable for the support layer to be made larger than the microneedle array.

The support layer may be made in a size and shape that make it easy to handle, depending on the application area, and for example, it may be preferable for the support layer to be larger than the microneedle array and protrude about 3 to 20 mm past an outer edge of the microneedle array. The thickness of the support layer may be the same as, thinner than, or thicker than the thickness of the substrate of the microneedle array, and may be made to have a thickness that allows the support layer to be flexible and thin to support the microneedle array, and to be easy to handle when in use.

The present invention may be used as a local anesthetic for dental treatments by appropriately setting the amount of local anesthetic included in the microneedle array per unit area and the size of the microneedle array. In addition, the present invention may also be used as a preanesthetic for relieving pain at an administration site before the local anesthetic for dental treatments is administered to the administration site. In this case, after the microneedle array and the local anesthesia patch of the present invention are attached to the oral mucosa or gums, the local anesthetic for dental treatments may be injected into the attachment site.

In this case, the substrate layer may also include the amide-based local anesthetic, the hyaluronic acid, and the polyvinylpyrrolidone, and in this case, the amide-based local anesthetic, the hyaluronic acid, and the polyvinylpyrrolidone may be included at a weight ratio ranging from 1:1.5:0.05 to 1:2.5:0.3, but the present invention is not limited thereto.

In the present invention, when the substrate layer is made of an amide-based local anesthetic, hyaluronic acid, and polyvinylpyrrolidone, the amide-based local anesthetic may be included at 30 to 34 wt %, the hyaluronic acid may be included at 61 to 65 wt %, and the polyvinylpyrrolidone may be included at 1 to 9 wt %. Preferably, the amide-based local anesthetic may be included at 31 to 33 wt %, the hyaluronic acid may be included at 62 to 64 wt %, and the polyvinylpyrrolidone may be included at 3 to 7 wt %, and most preferably, the amide-based local anesthetic may be included at 32 wt %, the hyaluronic acid may be included at 63 wt %, and the polyvinylpyrrolidone may be included at 5 wt %, but the present invention is not limited thereto.

In the present invention, the amide-based local anesthetic may preferably be lidocaine, and more preferably lidocaine hydrochloride.

When the microneedle or microneedle array according to the present invention is applied to the mucosa, gums, or skin, the dissolving microneedle may reach the inside of the mucosa, the inside of the gums, or the inside of the skin, and the amide-based local anesthetic acts as the microneedle part is dissolved.

The microneedle itself or the substrate of the microneedle array adheres closely to the curves of the mucosa, gums, or skin in a high-humidity environment within the mucosa, gums, or skin. When the microneedle array is applied, the local anesthetic contained in the substrate also enhances the local anesthetic effect.

When the microneedle array is applied to the oral cavity, gums, or skin, for the microneedle array to have an appropriate hardness that makes it difficult to bend the microneedle array and makes it easy for lidocaine to penetrate the oral cavity, gums, or skin, the microneedle array may be formed using a mixture of a high-molecular-weight polymer material with a weight average molecular weight of 100,000 or more and a low-molecular-weight polymer material with a weight average molecular weight of 50,000 or less. Preferably, the weight average molecular weight of the high-molecular-weight polymer material may be 50,000 or more and 2,000,000 or less. In addition, preferably, the weight average molecular weight of the low-molecular-weight polymer material may be 1,000 or more and 50,000 or less. In the present invention, the weight average molecular weight may be measured by gel permeation chromatography (GPC).

In another embodiment, the microneedle or microneedle array may be provided as a fine protrusion structure.

In the present invention, the fine protrusion structure, which is a fine protrusion structure for delivering a substance into the body, includes fine protrusions which are a first skin penetration site; and a microstructure which is a second skin penetration site and is coupled to tip portions of the fine protrusions, wherein the microstructure includes a main body formed of a biodegradable viscous composition and a coupling portion having a contact surface that extends from the main body to rear sides of the fine protrusions and comes in contact with outer side surfaces of the tip portions of the fine protrusions to allow the main body to be coupled to the fine protrusions and that has viscosity formed due to the biodegradable viscous composition, an inner layer and an outer layer of the microstructure are made of viscous compositions different from each other, the inner layer of the microstructure is made of a viscous composition having a relatively lower strength than the outer layer, the outer layer of the microstructure is made of a viscous composition having a relatively higher strength than the inner layer, the viscous compositions of the inner layer and the outer layer are made of at least two or more materials of hyaluronic acid and its salts, polyvinylpyrrolidone, cellulose polymer, dextran, gelatin, glycerin, polyethylene glycol, polysorbate, propylene glycol, povidone, and carbomer, gum ghatti, guar gum, glucomannan, glucosamine, dammer resin, rennet casein, locust bean gum, microfibrillated cellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin, pullulan, polyester, polyhydroxyalkanoates (PHAs), poly(α-hydroxy acid), poly(β hydroxy acid), poly(3-hydroxybutyrate-co-valerate) (PHBV), poly(3-hydroxypropionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyetherester, polyanhydride, poly(glycolic acid-co-trimethylene carbonate), polyphosphoester, polyphosphoester urethane, poly(amino acid), polycyanoacrylate, poly(trimethylene carbonate), poly(imino carbonate), poly(tyrosine carbonate), polycarbonate, poly(tyrosine arylate), polyalkylene oxalates, polyphosphazene, PHAPEG, chitosan, dextran, cellulose, heparin, hyaluronic acid, alginate, inulin, starch, or glycogen, and when the fine protrusions are separated from the skin in a state in which the microstructure is embedded in the skin, the coupling portion of the microstructure is separated from the fine protrusions in the inner layer having a lower strength than the outer layer, and thus the microstructure remains in the body. However, the present invention is not limited thereto.

The microneedle structure may deliver a substance into the body using a transdermal delivery device of the fine protrusion structure that includes (a) the fine protrusion structure; (b) a fixing means on which the fine protrusion structure is mounted; and (c) a guiding means that has an inner space accommodating the fixing means and guides the fine protrusion structure, which is mounted on the fixing means, to be applied to the skin, and in the transdermal delivery device of the fine protrusion structure, the guiding means may include an open upper part and a closed lower part, and a plurality of holes through which the fine protrusion structure passes may be formed in the closed lower part, but the present invention is not limited thereto.

The fine protrusion structure used in the present invention is described in detail in Korean Patent Publication No. 10-2016-0007923 (Korean Patent Registration No. 10-1703050), and although the detailed description thereof is omitted herein, the content described in the Korean publicized patent is incorporated by reference into the present specification as one embodiment for implementing the present invention.

### EXAMPLES

Hereinafter, the present invention will be described in more detail through examples. These examples are only for describing the present invention in more detail, and it will be apparent to those skilled in the art that the scope of the present invention is not limited by these examples according to the gist of the present invention.

### Example 1. Fabrication of Lidocaine-Containing Dissolving Microneedle (Li-DMN)

Hyaluronic acid (HA) (Bloomage Freda Biopharm Co. Ltd., Jinan, China) was used as an excipient for a Li-DMN, and polyvinylpyrrolidone (PVP) (BASF, Ludwigshafen, Rheinland-pfalz, Germany) was used as a disintegrant. To prepare a solution of drug and polymer, 15 wt % of lidocaine hydrochloride (Mahendra Chemicals, Gujarat, India), 31 wr % of HA, and 3 wt % of PVP were mixed with 51 wt % of distilled water, and the mixture was homogenized using a paste mixer (PDM-300C, KM TECH Co. Ltd., Icheon, Korea) to prepare a viscous solution (Li-HA solution). Then, using a robot dispenser (ML-5000X, Musashi Engineering, Inc., Tokyo, Japan), the Li-HA solution was applied in the form of viscous solution drops at 1.5 mm intervals in a hexagonal arrangement to a general-purpose polyurethane film. A single patch contained 2.11 mg of lidocaine hydrochloride and included 61 viscous solution drops. Using a centrifugal casting method which is a technique for fabricating a DMN using a centrifugal force, the viscous solution drops were centrifuged at 3,095×g rpm for 10 seconds to form a conical microneedle shape (Yang, H. et al., Adv. Healthc. Mater. 2017, 6, 1700326).

### Example 2. Performance Test

### 2-1. Microneedle Shape Test

To evaluate the morphological characteristics of the Li-DMN, microneedles were separated from three Li-DMN patches and then subjected to bright field microscopy (M165FC, Leica Camera AG, Wetzlar, Germany) and scanning electron microscopy (JSM-7610F Plus, JEOL Ltd., Tokyo, Japan), and microscopic images were acquired.

As a result, the length of the microneedles was confirmed to be 671.42±0.92 μm and the diameter was confirmed to be 31.65±8.90 μm, confirming that the length and the diameter satisfied the conditions of a length of 585 to 715 μm and a sharp end diameter of 50 μm or less, which are considered desirable for microneedles.

TABLE 1

| Item | Length (μm) | Diameter (μm) |
|---|---|---|
| Average value | 671.42 | 31.65 |
| Standard deviation | 30.92 | 8.90 |

### 2-2. Microneedle Strength Test

The analysis was performed using a mechanical tension and compression tester (OmniTest 5.0, Mecmesin Ltd, West Sussex, United Kingdom) for microneedles. After separating each DMN from three Li-DMN patches and placing each DMN on the stage of the tester, a primary peak value was measured for a load value measured when a 2 mm-diameter jig was lowered at a speed of 60 mm/s in the direction perpendicular to the microneedle using a tensile-compressive strength measuring device.

As a result, the strength was confirmed to be 83.1±38.3 mN in nine samples, and the strength of all samples was found to be higher than or equal to 0.020 N, which was the reference condition.

TABLE 2

| Sample | Peak (mN) |
|---|---|
| 1 | 59.576 |
| 2 | 164.178 |
| 3 | 89.941 |
| 4 | 88.419 |
| 5 | 40.022 |
| 6 | 61.933 |
| 7 | 45.157 |
| 8 | 87.413 |
| 9 | 111.099 |
| Average | 83.082 |
| Standard deviation | 38.321 |
| Pass rate | 100.0% |

### 2-3. Stickiness Test

The stickiness of Li-DMN patches was measured for three Li-DMN patches using the tension and compression tester (OmniTest 5.0, Mecmesin Ltd, West Sussex, United Kingdom) according to the Korean Pharmacopoeia guidelines.

After collecting ten samples and leaving them in a 37° C. thermostat for 30 minutes, a film was attached to one end of a test plate, and a rubber roller was immediately run over the film twice at an appropriate speed for about 1 minute. Then, the samples were left in the 37° C. thermostat for an additional 30 minutes. Half of the film was removed and fixed to the tension and compression device while being bent at 180°, and then a load value measured when pulling the film at a speed of 300 mm for 1 minute was recorded.

As a result, the stickiness was confirmed to be 119.48±28.49 gf/12 mm in the ten samples, and the stickiness of all samples was found to be higher than or equal to 42 gf/12 mm, which was the reference condition.

TABLE 3

| Sample | Stickiness (gf/12 mm) |
|---|---|
| 1 | 138.91 |
| 2 | 101.66 |
| 3 | 60.09 |
| 4 | 104.72 |
| 5 | 145.68 |
| 6 | 140.63 |
| 7 | 139.59 |
| 8 | 129.30 |
| 9 | 92.39 |
| 10 | 141.77 |
| Average | 119.48 |
| Standard deviation | 28.49 |
| Pass rate | 100.0% |

### 2-4. Content Test

A test solution was prepared by placing three Li-DMN patches in 12 mL of distilled water for 1 minute. 10 mL of a standard solution was prepared by precisely weighing 10 mg of standard lidocaine hydrochloride hydrate (1366013-150 MG, Sigma-Aldrich, St. Louis, MO, USA) and dissolving it by adding distilled water. 2 mL of trifluoroacetic acid (TFA) was mixed with 2,000 mL of water using an ultraviolet absorptiometer (Waters 2695, Waters, Milford, MA, USA), the mixture was used as mobile phase A, acetonitrile was used as mobile phase B, the mixture and acetonitrile were used while the ratio A:B was 70:30, and then lidocaine hydrochloride hydrate (C₁₄H₂₂N₂O·HCl·H₂O: 288.81) was detected at a measurement wavelength of 254 nm.

As a result, 1965.24±88.24 of lidocaine hydrochloride hydrate was confirmed in ten samples, the amount of lidocaine hydrochloride hydrate was confirmed to be 90.0 to 110.0% of the indicated amount in all ten samples, and thus all ten samples were confirmed to be suitable by the content test.

### 2-5. Dissolution Test

A dissolution test of Li-DMNs was performed using a dissolution tester (DIS 600i, Copley, Nottingham, United Kingdom) according to method 2 of the dissolution test method of the general test methods of the Korean Pharmacopoeia guidelines. Phosphate-buffered saline (PBS) was added to the dissolution tester, and the temperature of 37° C. and rotation speed of 100 rpm were maintained. Test samples were obtained by dissolving Li-DMN patches (n=6) in 900 mL of PBS. After 10 minutes, 10 mL test samples were collected and evaluated.

As a result, the dissolution amount was confirmed to be 1.682±0.005 mg in six samples, the average dissolution rate was confirmed to be 85.60%, the dissolution rate was confirmed to be 80% or higher in all six samples, and thus all six samples were confirmed to be suitable by the dissolution test.

### 2-6. Microbial Limit Test

The test was conducted according to the microbial limit test method of the general test methods of the Korean Pharmacopoeia guidelines. A test solution was prepared by placing each of ten patches in 90 mL of buffered sodium chloride-peptone solution (pH 7.0) and mixing them well. Using a petri dish with a diameter of 9 cm, 15 to 20 mL of Trypticase soy agar (TSA) or Sabouraud dextrose agar (SDA) at a temperature of about 45° C. was added to the petri dish and solidified, and then plate media were dried on a clean bench. Exactly 200 μl of the prepared test solution was taken and spread evenly over the entire surfaces of the media. The prepared test solution was tested using at least two petri dishes. Samples inoculated with TSA were cultured at 30 to 35° C. for 3 to 5 days, and samples inoculated with SDA were cultured at 20 to 25° C. for 5 to 7 days.

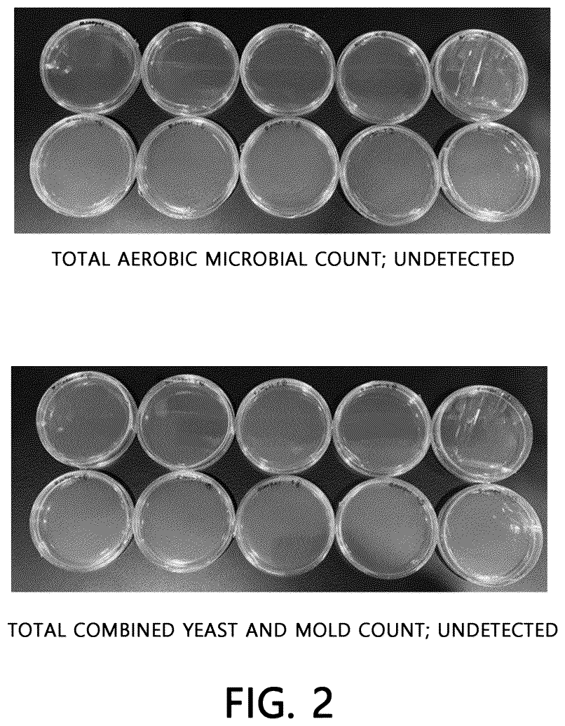

The total aerobic microbial count must be 1×100 CFU/g (mL) or less, the total combined yeast and mold count must be 10 CFU/g (mL) or less, and specific microorganisms (E. coli, Salmonella aeruginosa, and Staphylococcus aureus) must be judged as non-detectable, and since no aerobic microorganisms, yeasts, or molds were detected from the ten patches, the Li-DMN patches according to the present invention were confirmed to be suitable by the microbial limit test (FIG. 2).

### Example 3. Skin Irritation Test (Toxicity Test)

To confirm that the patch fabricated in Example 1 has no skin irritation, the skin reaction was evaluated after applying the patch to normal skin and abraded skin of six male New Zealand white rabbits (Sam: NZW, Samtako Bio Korea Co., Ltd.) for 24 hours and 72 hours (in accordance with Ministry of Food and Drug Safety Notice No. 2017-71).

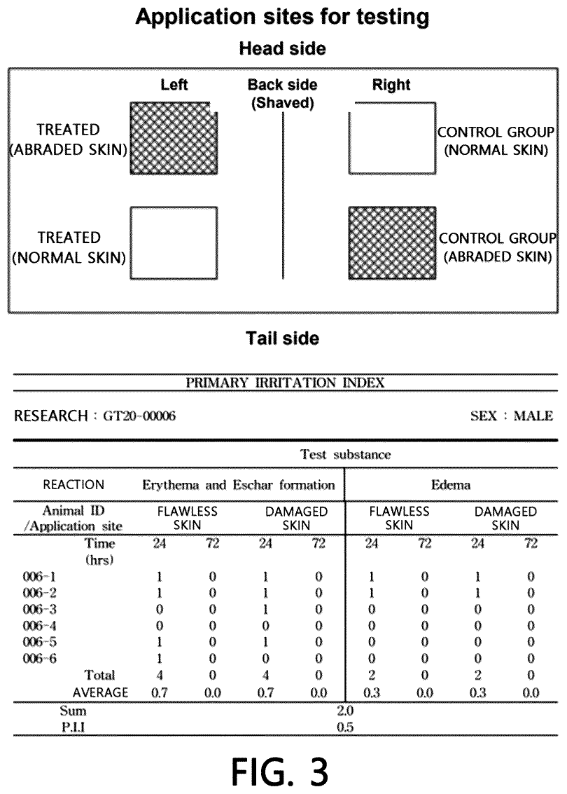

As a result of evaluating the skin reaction, the primary irritation index was calculated to be ‘0.5’ (FIG. 3), and thus the patch according to the present invention was found to be ‘non-irritating.’

### Example 4. Efficacy Test

The anesthetic efficacy of the patch fabricated in Example 1 was evaluated. Seven Sprague-Dawley male 6-week-old rats (Crlj:CD(SD), Coretech Co., Ltd.) were used per group as test animals, the patch was attached to the paws of the rats for 10 minutes and then removed, and the anesthetic efficacy was evaluated at 0 minutes, 10 minutes, and 20 minutes after the patch was removed.

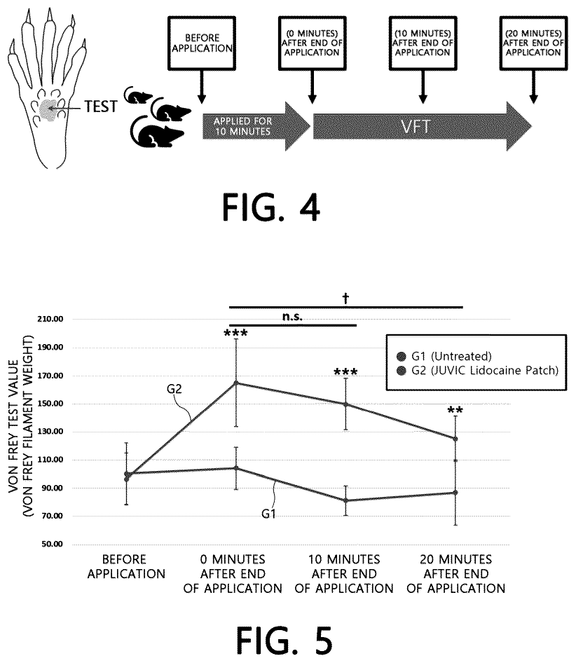

When mechanical stimulation was applied to the paws in a manner in which the intensity was gradually increased, the time during which the feet suddenly avoided or withdrew from filament stimulation was defined as a positive response, and a filament strength at that time was measured. For accurate measurements, pressure was applied until a von Frey filament was slightly bent (FIG. 4).

As a result of analyzing the threshold level by performing a von Frey test at 0 minutes, 10 minutes, and 20 minutes after application of the patch, the threshold level of a test group (G2, JUVIC Lidocaine Patch) to which the patch of the present invention was attached was always found to be higher than the threshold level of an untreated group (G1) (FIG. 5).

### Example 5. Administration Dose Evaluation

In order to confirm the minimum dose at which the anesthetic effect of the patch according to the present invention is achieved, the anesthetic effect according to the administration dose was confirmed using a pig jawbone.

Five pieces of pig jawbone (Cronex Co., Ltd.) were used per group for the test, patches fabricated in the same manner as in Example 1 to contain three different doses, 1 mg, 1.5 mg, and 2 mg, of lidocaine were used for a test group, no treatment was used for a negative control group, and Xogel Adult Gingival Gel (hereinafter referred to as “Xogel,” Shinwon Dental Co., Ltd.) was used for a positive control group.

For the test group, the patches were applied to the gum area for 3 minutes and then removed, and for the positive control group, 0.1 g of the gel (containing 5 mg of lidocaine), which is the actual clinical dose, was applied. The oral tissue at the area where the drug was applied was biopsied and extracted, 200 μL of methanol was added to a gum area of the extracted oral tissue, the oral tissue was crushed in a homogenizer for 30 minutes and centrifuged at 18,000×g for 7 minutes, and a supernatant was extracted to analyze the concentration of lidocaine delivered to the tissue through liquid chromatography-mass spectrometry (LC-MS) or mass spectrometry (MS).

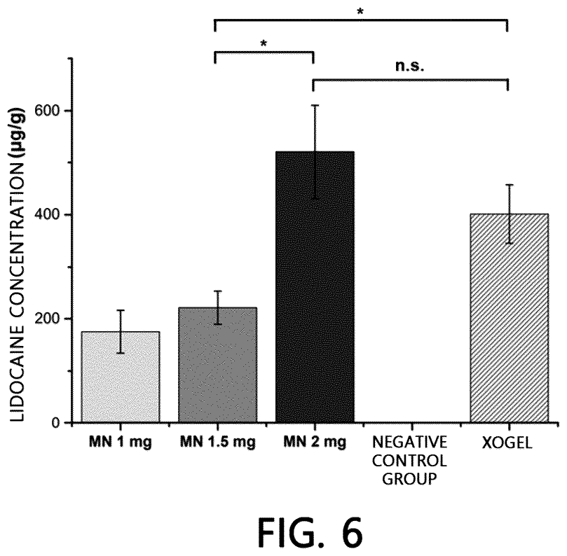

As a result, as shown in FIG. 6, while the concentration of lidocaine delivered locally to the oral cavity after Xogel was applied was found to be 401.35±55.89 μg/g, the concentration of lidocaine delivered to the oral cavity after the patch according to the present invention (containing 2 mg of lidocaine) was applied was found to be 520.51±89.88 μg/g, and thus it was confirmed that when the patch according to the present invention was used, the effect was better than Xogel even when lidocaine was used at 40% dose compared to lidocaine used in Xogel. However, when the patch according to the present invention containing 1 mg or 1.5 mg of lidocaine was used for 3 minutes, the concentration of the drug in the oral tissue was statistically significantly lower as compared to when Xogel was used.

Therefore, it was confirmed that the patch according to the present invention had a relatively better clinical effect than Xogel when formulated to contain 2 mg of lidocaine.

Although specific parts of the present invention have been described above in detail, it will be apparent to those skilled in the art that these specific techniques are merely a preferred embodiment and that the scope of the present invention is not limited thereto. Therefore, the substantial scope of the present invention will be defined by the accompanying claims and their equivalents.

The present invention was supported by the following national research and development project.
[Task identification number] 1465033263
[Task number] HI16C0625
[Name of ministry] Ministry of Health and Welfare
[Name of (specialized) institute in charge of task management] Korea Health Industry Development Institute
[Research project name] Advanced Medical Technology Development (R&D)
[Research task name] Development of biodegradable microstructure technology-based local anesthesia film
[Name of institute in charge of task performance] JUVIC Co., Ltd.
[Research period] Apr. 1, 2016-Dec. 31, 2022

1-10. (canceled)

11. A local anesthesia patch comprising:
(i) a dissolving microneedle comprising an amide-based local anesthetic, hyaluronic acid and polyvinylpyrrolidone; and
(ii) a support layer configured to support the microneedle,
wherein the amide-based local anesthetic is one or more selected from the group consisting of lidocaine, mepivacaine, prilocaine, articaine, bupivacaine, etidocaine, and salts thereof,
wherein the amide-based local anesthetic, the hyaluronic acid, and the polyvinylpyrrolidone are included at a weight ratio ranging from 1:1.5:0.05 to 1:2.5:0.3,
wherein the support layer has a thickness ranging from 150 to 500 μm
wherein the microneedle has a height ranging from 500 to 1,000 μm,
wherein the support layer is a polymer film and is:
(i) a plastic sheet or film selected from the group consisting of polyethylene, polyurethane, polyvinyl alcohol, polypropylene, polyethylene terephthalate, polystyrene (GPPS), polylactide, polyglycolide, polycaprolactone, polyethylene glycol, polyanhydride, polyamide, polyesteramide, polyorthoester, polydioxanone, polyacetal, polyketal, polycarbonate, polyorthocarbonate, polyphosphazene, polyhydroxybutyrate, polyhydroxyvalerate, polyalkylene oxalates, polyalkylene succinates, poly(malic acid), poly(amino acid), poly(methylvinyl ether), chitin, chitosan and its copolymer, terpolymer, carboxymethylcellulose, hyaluronic acid, polyvinylpyrrolidone, and an ethylene vinyl acetate (EVA) copolymer;
(ii) a paper sheet selected from the group consisting of sterilization paper, cellophane, non-woven fabric, and woven fabric;
(iii) a silicone resin thin film formed by spraying or spreading; or
(iv) a fluorine oil thin film formed by spraying or spreading.

12-13. (canceled)

14. The local anesthesia patch of claim 11, wherein the local anesthesia patch contains 1 mg to 5 mg of lidocaine.

15. (canceled)

16. The local anesthesia patch of claim 11, wherein the amide-based local anesthetic, the hyaluronic acid, and the polyvinylpyrrolidone are included at 32 wt %, 63 wt %, and 5 wt %, respectively.

17. The local anesthesia patch of claim 11, wherein the local anesthesia patch is for local anesthesia for treating dental or dermatological diseases.

18. The local anesthesia patch of claim 11, wherein the dissolving microneedle is conical, candle-shaped, or egg-shaped.

19. The local anesthesia patch of claim 11, wherein the dissolving microneedle is made of three layers consisting of a support portion, a middle end portion, and a sharp end portion, and the amide-based local anesthetic, the hyaluronic acid, and the polyvinylpyrrolidone are included in the middle end portion.

20. The local anesthesia patch of claim 19, wherein the amide-based local anesthetic, the hyaluronic acid, and the polyvinylpyrrolidone constitute an entire layer of the middle end portion.

21. The local anesthesia patch of claim 19, wherein the amide-based local anesthetic, the hyaluronic acid, and the polyvinylpyrrolidone are located in a core layer of the middle end portion, and the core layer is formed to be covered by a shell layer.

22. A method for providing local anesthesia, the method comprising applying the local anesthesia patch of claim 11 to a skin of a subject in need thereof.
